(19) United States
(12) Patent Application Publication
(10) Pub. No.: US 2015/0374034 A1
(43) Pub. Date: Dec. 31, 2015

(54) Nicotine Dosage Regimen

(71) Applicant: KIND CONSUMER LTD, London (GB)

(72) Inventors: Alex Hearn, London (GB); Chris Moyses, Oxford (GB); Ritika Gupta, London (GB)

(21) Appl. No.: 14/747,572

(22) Filed: Jun. 23, 2015

Related U.S. Application Data
(60) Provisional application No. 62/016,861, filed on Jun. 25, 2014.

(51) Int. Cl. A24F 47/00; A61K 31/465; A61K 9/00

(52) U.S. Cl. CPC A24F 47/002; A61K 9/008; A61K 31/465

(57) ABSTRACT

The invention relates to dosage regimens for an inhalable formulation comprising nicotine, and to devices for delivering said dosage regimens.

The invention relates to dosage regimens for an inhalable formulation comprising nicotine, and to devices for delivering said dosage regimens.

The smoking of tobacco is an addictive activity associated with the pleasurable feeling caused by nicotine, and reinforced by the habits and rituals of the smoker. These attributes combine to make it very difficult to give up smoking, despite the numerous adverse health effects of the carbon monoxide, tar, and other combustion products of tobacco. It is not the nicotine itself that is harmful to health, rather the by-products of tobacco smoke.

There are a number of smoking cessation aids currently on the market for use in effective nicotine replacement therapy (NRT), such as nicotine skin patches, nicotine-containing gums, nicotine cartridges, and nicotine inhalers. These aids attempt to achieve the increase in blood nicotine content provided by tobacco smoke without the associated dangerous by-products. Among the various modes of NRT, nicotine inhalers most closely replicate the rituals of smoking. A class of nicotine inhalers are termed ‘vaporizers’ or ‘electronic cigarettes’. In electronic “e”-cigarettes, as is the case in conventional tobacco cigarettes, nicotine must be heated in order to be delivered orally to a user (to result in combustion in the case of a conventional cigarette or to result in vaporisation in the case of an e-cigarette). Such heating results in the generation of harmful by-products, such as aldehydes, ketones, nitrosamines and heavy metals, which are then also delivered to the user via inhalation. Thus, there are potential health consequences of using e-cigarettes as NRT.

Nicotine inhalers which do not require vaporization are available on the market. These inhalers provide a nicotine formulation in a porous plug of polyethylene, for example the Nicorette® Inhalator. Porous plug inhalers are available in 10 mg or 15 mg cartridges, however the delivered dose of nicotine is significantly lower than the quantity provided in the cartridge. This is because in normal use, much of the nicotine remains adsorbed to the polyethylene plug. For example, on average, 4 mg nicotine is released from a 10 mg cartridge following 20 minutes of intensive use (Hukkanen et al., Pharmacol. Rev. 2005, 57, 79). However, the distribution of delivered nicotine dose from a porous plug inhaler cartridge is highly variable. Delivered doses range from 1.3 mg to 6.2 mg per cartridge. Furthermore, the nicotine release profile from the polyethylene plug is highly temperature dependant. The delivered dose increases by 35% for each 10° C. increase in temperature.

Owing to the pharmacokinetic profile of nicotine when delivered to a user by porous plug inhalers, inhalers of such type require a dosage regimen of up to 12 a day of the 10 mg cartridges, or up to 6 a day of the 15 mg cartridges, in order to relieve craving associated with nicotine dependence. The use of such high strength nicotine formulations at such high frequency raises safety concerns. Given the high variability of the delivered nicotine dose, and its temperature dependence, there is a risk that use of a porous plug inhaler in high temperature environments, for example at temperatures of 37° C. or more, under the dosage regimen required to relieve craving can lead to a total daily delivered dose of greater than 60 mg. Such high levels of daily nicotine intake run the risk of exposing the user to toxic or even lethal doses of nicotine: the minimal lethal dose of nicotine is reported to be as low as 30 mg. This risk is exacerbated in users with impaired nicotine metabolism. Furthermore, the use of high and inconsistent doses of nicotine provided by porous plug inhalers can result in high nicotine plasma concentrations, ultimately resulting in prolonged nicotine dependence and low success in smoking cessation.

There exists a need to provide a dosing regimen for inhalable NRT that overcomes problems inherent in the prior art.

The present invention provides improved formulations, therapeutic applications and dosage regimens for use in NRT. Thus, the present invention provides a dosing regimen comprising a daily dose of an inhalable nicotine formulation for use in (1) a method of relieving or preventing nicotine craving associated with tobacco dependence in a subject, or (2) a method of relieving or preventing withdrawal symptoms associated with tobacco dependence in a subject, or (3) a method of reducing or preventing consumption of inhaled tobacco smoke in a subject, wherein the daily dose comprises a deliverable daily dose of less than 60 mg of nicotine or a pharmaceutically acceptable salt thereof.

In one embodiment, the invention provides an orally inhalable formulation comprising nicotine or a pharmaceutically acceptable salt thereof and a propellant for use in a method of relieving or preventing nicotine craving associated with tobacco dependence in a subject, wherein a daily dose of inhalable formulation is provided in one or more pressurized containers, wherein the inhalable formulation is for pulmonary administration using a simulated cigarette, said simulated cigarette having a housing, a reservoir configured to receive and contain a charge of inhalable formulation from the pressurized container, and an outlet valve, and wherein said daily dose of inhalable formulation comprises a deliverable daily dose of less than 60 mg of nicotine or a pharmaceutically acceptable salt thereof.

Each aspect or embodiment as defined herein may be combined with any other aspect(s) or embodiment(s) unless clearly indicated to the contrary. In particular any feature indicated as being preferred or advantageous may be combined with any other feature or features indicated as being preferred or advantageous.

It has been surprisingly found that inhalable nicotine formulations according to the present invention deliver a highly consistent dose of nicotine to the user, and when used in the manner of the present invention are capable of relieving or preventing nicotine craving associated with tobacco dependence using lower doses of nicotine than in known orally inhalable NRT modes. When administered according to the dosing regimen of the present invention, the presently claimed formulations are capable of replicating many of the rituals associated with smoking, which provides a psychological boost to the physiological effects of the administered nicotine. This enables effective relief of nicotine craving or withdrawal symptoms associated with tobacco dependence using lower quantities of nicotine than are required to achieve the same level of relief in dosage regimens known for existing orally inhalable nicotine formulations.

Furthermore, the dose delivered by each inhalation of the present dosage regimen is generally uniform, and is temperature independent. Thus, it is possible to provide a dosage regimen that is sufficient to relieve or prevent nicotine cravings without exposing the user to potentially deliverable dose of nicotine in excess of 60 mg per day.

In another aspect of the present invention, an orally inhalable formulation is provided comprising nicotine or a pharmaceutically acceptable salt thereof and a propellant for use in a method of relieving or preventing withdrawal symptoms associated with tobacco dependence in a subject, wherein a daily dose of inhalable formulation is provided in one or more pressurized containers, wherein the inhalable formulation is for pulmonary administration using a simulated cigarette, said simulated cigarette having a housing, a reservoir configured to receive and contain a charge of inhalable formulation from the pressurized container, and an outlet valve, and wherein said daily dose of inhalable formulation comprises a deliverable daily dose of less than 60 mg of nicotine or a pharmaceutically acceptable salt thereof.

In another aspect of the present invention, an orally inhalable formulation is provided comprising nicotine or a pharmaceutically acceptable salt thereof and a propellant for use in a method of reducing or preventing consumption of inhaled tobacco smoke in a subject, wherein a daily dose of inhalable formulation is provided in one or more pressurized containers, wherein the inhalable formulation is for pulmonary administration using a simulated cigarette, said simulated cigarette having a housing, a reservoir configured to receive and contain a charge of inhalable formulation from the pressurized container, and an outlet valve, and wherein said daily dose of inhalable formulation comprises a deliverable daily dose of less than 60 mg of nicotine or a pharmaceutically acceptable salt thereof.

In a further embodiment of the invention is a method of relieving or preventing nicotine craving associated with tobacco dependence in a subject, said method comprising the step of administering to the subject an orally inhalable formulation comprising nicotine or a pharmaceutically acceptable salt thereof and a propellant, wherein a daily dose of inhalable formulation is provided in one or more pressurized containers,

wherein the inhalable formulation is for pulmonary administration using a simulated cigarette, said simulated cigarette having a housing, a reservoir configured to receive and contain a charge of inhalable formulation from the pressurized container, and an outlet valve, and wherein said daily dose of inhalable formulation comprises a deliverable daily dose of less than 60 mg of nicotine or a pharmaceutically acceptable salt thereof.

In a further embodiment of the invention is a method of relieving or preventing withdrawal symptoms associated with tobacco dependence in a subject, said method comprising the step of administering to the subject an orally inhalable formulation comprising nicotine or a pharmaceutically acceptable salt thereof and a propellant,

wherein a daily dose of inhalable formulation is provided in one or more pressurized containers, wherein the inhalable formulation is for pulmonary administration using a simulated cigarette, said simulated cigarette having a housing, a reservoir configured to receive and contain a charge of inhalable formulation from the pressurized container, and an outlet valve, and wherein said daily dose of inhalable formulation comprises a deliverable daily dose of less than 60 mg of nicotine or a pharmaceutically acceptable salt thereof.

In a further embodiment of the invention is a method of reducing or preventing consumption of inhaled tobacco smoke in a subject, said method comprising the step of administering to the subject an orally inhalable formulation comprising nicotine or a pharmaceutically acceptable salt thereof and a propellant, wherein a daily dose of inhalable formulation is provided in one or more pressurized containers, wherein the inhalable formulation is for pulmonary administration using a simulated cigarette, said simulated cigarette having a housing, a reservoir configured to receive and contain a charge of inhalable formulation from the pressurized container, and an outlet valve, and wherein said daily dose of inhalable formulation comprises a deliverable daily dose of less than 60 mg of nicotine or a pharmaceutically acceptable salt thereof.

The term “diameter” as used herein encompasses the largest dimension of a droplet. Droplet diameters referred to herein may be measured using a Malvern Spraytec device.

The term “Dv10” as used herein refers to a droplet diameter that no more than 10% vol of the droplets in a formulation have a smaller diameter than. The term “Dv50” as used herein refers to a droplet diameter that no more than 50% vol of the droplets in a formulation have a smaller diameter than. The term “Dv90” as used herein refers to a droplet diameter that no more than 90% vol of the droplets in a formulation have a smaller diameter than. Dv10, Dv50 and Dv90 values may be determined using a Malvern Spraytec device.

The term “nicotine free base” as used herein refers to the form of nicotine that predominates at high pH levels, i.e. at pH levels above 7.

The term “Cₘₐₓ” as used herein refers to the maximum measured concentration of a compound, in this case nicotine, in the bloodstream of a subject.

The term “tₘₐₓ” as used herein refers to the time taken to achieve Cₘₐₓ from administration of the compound.

The terms “patient”, “subject” and “user” are used interchangeably herein, and refer to an animal, preferably a human, to whom NRT is applied.

The term “deliverable daily dose” means the cumulative amount of nicotine that is pulminarily administered to the user over the course of a 24 hour period. According to aspects of the present invention the inhalable formulation is released from the pressurized container into the reservoir of a simulated cigarette by action of a pressure gradient between the two devices. The deliverable daily dose of nicotine is the amount of nicotine that can be transferred from the pressurized container to the user via iterative charges of an empty reservoir of a simulated cigarette, accounting for escape of formulation during refills.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

According to one embodiment of the present invention, the total content of nicotine or a pharmaceutically acceptable salt thereof of the daily dose contained in the one or more pressurized containers is 0.2 mg or more and does not exceed 75 mg, 70 mg, 65 mg, 60 mg, 55 mg, 50 mg, 45 mg, 40 mg, 35 mg, 30 mg, 25 mg, 20 mg, 19 mg, 18 mg, 17 mg, 16 mg, 15 mg, 14 mg, 13 mg, 12 mg, 11 mg, 10 mg, 9.5 mg, 9 mg, 8.5 mg, 8 mg, 7.5 mg, 7 mg, 6.5 mg, 6 mg, 5.5 mg, 5 mg, 4.5 mg 4 mg, 3.5 mg, 3 mg, 2.5 mg, 2 mg, 1.5 mg, 1 mg, 0.9 mg, 0.8 mg, 0.7 mg, 0.6 mg, 0.5 mg, 0.45 mg, 0.4 mg, 0.35 mg, 0.3 mg or 0.25 mg.

In an embodiment of the invention, the deliverable daily dose of nicotine or a pharmaceutically acceptable salt thereof is at least 60%, preferably at least 70%, more preferably at least 75% of the total dose of nicotine or pharmaceutically acceptable salt thereof contained in the inhalable formulation provided in the one or more pressurized containers.

In an aspect of the present invention, the daily dose of inhalable formulation comprises a deliverable daily dose that is 0.2 mg or more, and does not exceed 60 mg, 55 mg, 50 mg, 45 mg, 40 mg, 35 mg, 30 mg, 25 mg, 20 mg, 19 mg, 18 mg, 17 mg, 16 mg, 15 mg, 14 mg, 13 mg, 12 mg, 11 mg, 10 mg, 9.5 mg, 9 mg, 8.5 mg, 8 mg, 7.5 mg, 7 mg, 6.5 mg, 6 mg, 5.5 mg, 5 mg, 4.5 mg, 4 mg, 3.5 mg, 3 mg, 2.5 mg, 2 mg, 1.5 mg, 1 mg, 0.9 mg, 0.8 mg, 0.7 mg, 0.6 mg, 0.5 mg, 0.45 mg, 0.4 mg, 0.35 mg, 0.3 mg, 0.25 mg or 0.2 mg. In a preferred embodiment, the deliverable daily dose is 40 mg or less, preferably 20 mg or less, more preferably 18 mg or less.

A particular advantage of formulations, devices, administration modes and regimens of the present invention is that they do not contain or produce harmful chemicals known to occur in tobacco smoke, which in addition to being toxic the user, can also be extremely harmful to unborn foetuses or breast-feeding infants. Thus, the formulations, devices, administration modes and regimens described herein provide an effective treatment of nicotine craving associated with tobacco dependence in a subject who is either pregnant of lactating. Accordingly, in an embodiment of the invention is provided the formulations, devices, administration modes and dosage regimens described herein for use in a method of relieving or preventing nicotine craving associated with tobacco smoke in a pregnant subject or in a lactating subject. In an alternative embodiment of the invention is provided the formulations, devices, administration modes and dosage regimens described herein for use in a method of relieving or preventing withdrawal symptoms associated with tobacco smoke in a pregnant subject or in a lactating subject. In an alternative embodiment of the invention is provided the formulations, devices, administration modes and dosage regimens described herein for use in a method of reducing or preventing consumption of inhaled tobacco smoke in a pregnant subject or in a lactating subject.

In an embodiment of the invention, the subject is free to self-administer a dose that is lower than the daily dose provided herein, and the daily dose described herein is a maximum daily dose.

In an aspect of the present invention, the deliverable dose of nicotine or a pharmaceutically acceptable salt thereof is substantially temperature independent. In an embodiment of the invention, the deliverable dose remains constant across a temperature range of from about 15° C. to about 50° C., preferably from about 20° C. to about 30° C. Accordingly, in an embodiment of the invention the methods and dosage regimens of the present invention are administered at a temperature of 50° C. or less, preferably 37° C. or less, more preferably 30° C. or less.

In embodiments of the present invention, a pressurized container may comprises from about 2 mg to about 30 mg, or about 2 mg to about 25 mg, or about 2 mg to about 20 mg, or about 3 mg to about 19 mg, or about 4 mg to about 18 mg, or about 5 mg to about 17 mg, or about 6 mg to about 16 mg, or about 7 mg to about 15 mg or about 8 mg to about 14 mg, or about 8 mg to about 13 mg, or about 8 mg to about 12 mg, or about 8 mg to about 11 mg, or about 9 mg to about 10 mg nicotine or a pharmaceutically acceptable salt thereof. In preferred embodiments, the pressurized container may comprise about 15 mg to about 20 mg, preferably about 16 mg to about 18 mg, more preferably about 17 mg to about 18 mg nicotine or a pharmaceutically acceptable salt thereof. In an alternative preferred embodiment, the pressurized container may comprise from about 7 mg to about 14 mg, preferably about 8 mg to about 13 mg, more preferably about 11 mg to about 12 mg nicotine or a pharmaceutically acceptable salt thereof. In an alternative preferred embodiment, the pressurized container may comprise from about 2 mg to about 7 mg, preferably about 3 mg to about 6 mg, more preferably from about 4 mg to about 5 mg nicotine or a pharmaceutically acceptable salt thereof.

According to the present invention, the deliverable dose of nicotine or a pharmaceutically acceptable salt thereof provided by the pressurized container is at least 60%, preferably at least 70%, more preferably at least 75%, more preferably at least 78% of the total amount of nicotine or pharmaceutically acceptable salt thereof contained in the pressurized containers.

In particularly preferred embodiments, the pressurized container comprises from about 16 mg to about 18 mg nicotine or a pharmaceutically acceptable salt thereof and provides a deliverable dose of at least 75%, preferably at least 78% of the total amount of nicotine or pharmaceutically acceptable salt thereof contained in the pressurized container. In alternative preferred embodiments, the pressurized container comprises from about 11 mg to about 12 mg nicotine or a pharmaceutically acceptable salt thereof and provides a deliverable dose of at least 75%, preferably at least 78% of the total amount of nicotine or pharmaceutically acceptable salt thereof contained in the pressurized container. In alternative preferred embodiments, the pressurized container comprises from about 4 mg to about 5 mg nicotine or a pharmaceutically acceptable salt thereof and provides a deliverable dose of at least 75%, preferably at least 78% of the total amount of nicotine or pharmaceutically acceptable salt thereof contained in the pressurized container.

The one or more pressurized containers may consist of the number of pressurized containers necessary for a delivered daily dose of inhalable formulation according to the present invention. In a preferred embodiment of the invention, the total nicotine content is such that the delivered daily dose of the present invention is provided in two pressurized containers. In an alternative preferred embodiment, the pressurized containers comprise from about 2 mg to about 7 mg nicotine or a pharmaceutically acceptable salt thereof, and the delivered daily dose of inhalable formulation is provided in five pressurized containers, or alternatively in four pressurized containers.

In an embodiment of the present invention, the pressurized container comprises from about 5 charges to about 40 charges, wherein a charge is an amount of inhalable formulation necessary to fill the reservoir of a simulated cigarette. In a preferred embodiment the pressurized container comprises from about 10 charges to about 35 charges, or about 15 charges to about 30 charges, or about 17 charges to about 25 charges of inhalable formulation. In a preferred embodiment the pressurized container comprises approximately 20 charges of inhalable formulation.

In one embodiment of the present invention each charge comprises approximately 0.1 mg-1 mg nicotine or a pharmaceutically acceptable salt thereof, or approximately 0.2 mg-0.9 mg, or approximately 0.3 mg-0.8 mg, or approximately 0.3 mg-0.7 mg, or approximately 0.4 mg-0.6 mg, or approximately 0.4 mg-0.5 mg, or approximately 0.4-0.6 mg nicotine or a pharmaceutically acceptable salt thereof. In a preferred embodiment each charge comprises approximately 0.66 mg-0.69 mg nicotine or a pharmaceutically acceptable salt thereof, or approximately 0.43 mg-0.45 mg nicotine or a pharmaceutically acceptable salt thereof, or approximately 0.21 mg-0.23 nicotine or a pharmaceutically acceptable salt thereof.

In one embodiment of the present invention, the first charge administered to a simulated cigarette according to the present invention comprises less nicotine than the second and subsequent charges. Thus, in an embodiment of the invention, the first charge comprises less than approximately 1 mg nicotine or a pharmaceutically acceptable salt thereof, preferably less than approximately 0.43 mg, more preferably approximately 0.02-0.3 mg. In these embodiments, the second and subsequent charges may comprise approximately 0.1 mg-1 mg nicotine or a pharmaceutically acceptable salt thereof, or approximately 0.2 mg-0.9 mg, or approximately 0.3 mg-0.8 mg, or approximately 0.3 mg-0.7 mg, or approximately 0.4 mg-0.6 mg, or approximately 0.4 mg-0.5 mg, or approximately 0.4-0.6 mg nicotine or a pharmaceutically acceptable salt thereof, provided that the nicotine content of the first charge is less than the second and subsequent charges. In a preferred embodiment the first charge comprises less nicotine or a pharmaceutically acceptable salt thereof than the second and subsequent charges, wherein the second and subsequent charges comprise approximately 0.66 mg-0.69 mg nicotine or a pharmaceutically acceptable salt thereof, or approximately 0.43 mg-0.45 mg nicotine or a pharmaceutically acceptable salt thereof, or approximately 0.21 mg-0.23 nicotine or a pharmaceutically acceptable salt thereof.

In a preferred embodiment of the invention, each charge contains approximately 5-15 inhalations, preferably approximately 6-12 inhalations, more preferably approximately 7-10 inhalations, more preferably about 8 inhalations or about 9 inhalations. In a preferred embodiment each charge is consumed at a rate of approximately one inhalation per 0.05 minutes to one inhalation per 2 minutes, preferably approximately one inhalation per 0.1 minutes to one inhalation per 1 minutes, preferably approximately one inhalation per 0.2 minutes to one inhalation per 0.5 minutes, more preferably approximately one inhalation per 0.25 minutes. In a preferred embodiment of the invention each charge is consumed over a period of up to 10 minutes, or up to 6 minutes, or up to 5 minutes, and preferably up to 4 minutes, more preferably up to 3 minutes and more preferably up to 2 minutes.

In preferred embodiments of the invention, the orally inhalable formulation comprises:
nicotine or a pharmaceutically acceptable derivative or salt thereof;
a propellant;
a monohydric alcohol; and
a glycol and/or glycol ether,
   characterised in that the ratio of monohydric alcohol:glycol and/or glycol ether by weight is from 6:1 to 1:1.

The glycol and/or glycol ether aids the dissolution of the nicotine or a pharmaceutically acceptable derivative or salt thereof in the formulation. This avoids the presence of precipitates of nicotine (or other additives such as saccharin, if present) in the formulation, which could cause irritation when delivered to a user. In addition, the presence of glycol or glycol ether reduces the degradation of nicotine that occurs over time, thereby increasing the long-term stability or “shelf life” of the formulation.

Monohydric alcohol has a lower viscosity than a glycol or glycol ether. Accordingly, the formulation is able to form droplets of a smaller diameter in comparison to formulations in which the monohydric alcohol is not present. The present inventors have surprisingly found that the ratio of monohydric alcohol to glycol or glycol ether specified above results in a formulation with a desired combination of both long term stability (for example the formulation remains as a single phase for at least a week at a temperature of 2-40° C.) and small droplet size.

Advantageously, when a nicotine formulation having such a ratio of monohydric alcohol:glycol or glycol ether is delivered to a user via a conventional pressurised metered-dose inhaler (pMDI), the formulation is delivered in the form of droplets, some of which (such as, for example, at least 10% vol) have a diameter of less than 10 μm, typically less than 5 μm. Typically, the majority (such as, for example, at least 50% vol) of the droplets have a diameter of less than 5 μm, typically substantially all (such as, for example, at least 90% vol, or even at least 95% vol) of the droplets have a diameter of less than 5 μm. Advantageously, when administered orally, droplets with a size of less than 10 μm tend to be deposited in the lungs, rather than, for example, the oropharynx. Accordingly, at least some (such as, for example, at least 10% w/w), typically substantially all (such as, for example, at least 90% w/w), of the nicotine enters the bloodstream via the pulmonary route. This means that the formulation, when inhaled orally, is more able to mimic the pharmacokinetic profile of a conventional cigarette compared to nicotine formulations of the prior art. Since the formulation may be administered orally and is able to mimic the pharmacokinetic profile of a conventional cigarette, it is particularly effective for use in NRT or as an alternative to recreational smoking of conventional cigarettes.

Typically at least some (such as, for example, at least 10% vol) of the droplets have a size of from 0.5 to 3 μm. Such droplets may be deposited in the deep lung, and are therefore particularly able to enter the blood stream via the pulmonary route. Typically at least some (such as, for example, at least 10% vol) of the droplets have a diameter of from 0.4 to 0.5 μm. Such droplets are particularly able to mimic the pharmacokinetic profile of a conventional cigarette, since conventional cigarette smoke has a mean particle diameter in the range of from 0.4 to 0.5 μm.

When the formulation of the present invention is delivered to a user via one of the simulated cigarettes described below, the droplets may exhibit the following droplet size profile:
Dv 90 of less than 20 μm, typically less than 5 μm, more typically less than 3, even more typically less than 2.9 μm, and/or
Dv 50 of less than 6 μm, typically less than 0.8 μm, more typically less than 0.7 μm, even more typically less than 0.6 μm, and/or
Dv 10 of less than 2 μm, typically less than 0.3 μm, more typically less than 0.25 μm, even more typically less than 0.2 μm.

This particular droplet size profile is similar to the particle size profile of tobacco smoke. Accordingly, the pharmacokinetic profile of the delivered formulation closely mimics that of a conventional cigarette. In particular, delivery of the formulation to a user generates an extended peak of high nicotine concentration with a short tₘₐₓ, i.e. the time from first inhalation to the maximum nicotine-plasma level. As a result, the formulation is highly effective for use in NRT and is capable of effectively relieving nicotine craving associated with tobacco dependence or withdrawal symptoms associated with tobacco dependence at lower deliverable doses of nicotine than other orally inhalable modes of NRT.

Any suitable source of nicotine may be employed. For example, the nicotine may be nicotine free base, a nicotine derivative and/or a nicotine salt. Where a nicotine free base is employed, it may be employed in liquid form. Where a nicotine salt is employed, it may be employed in the form of a solution. Suitable nicotine salts include salts formed of the following acids: acetic, proprionic, 1,2-butyric, methylbutyric, valeric, lauric, palmitic, tartaric, citric, malic, oxalic, benzoic, alginic, hydrochloric, chloroplatinic, silicotungstic, pyruvic, glutamic and aspartic. Other nicotine salts, such as nicotine bitartrate dehydrate, may also be employed. Mixtures of two or more nicotine salts may be employed. Nicotine salts may also be in liposomal encapsulation. Such encapsulation may allow the nicotine concentration of a formulation to be further increased without nicotine precipitation occurring. As used herein the weight of nicotine or a pharmaceutically acceptable salt thereof refers to the free-base form of nicotine. Therefore, when a nicotine salt is employed, the molar equivalent to the free-base weight should be calculated.

As discussed above, the ratio of monohydric alcohol:glycol or glycol ether by weight results in a combination of both stability and a desired droplet size profile. Preferably the ratio of monohydric alcohol:glycol or glycol ether by weight is from 5:1 to 1.5:1, preferably from 4:1 to 2:1, more preferably from 3:1 to 2.5:1, even more preferably about 2.8:1.

The glycol and/or glycol ether may be selected from propylene glycol, polypropylene glycol and polyethylene glycol (PEG), or combinations of two or more thereof. Suitably polyethylene glycols may have a molecular mass of less than 20,000 g/mol. An example of a suitable polyethylene glycol is PEG 400. Preferably the glycol or glycol ether is propylene glycol. Propylene glycol provides the formulation with a particularly desirable droplet size profile and provides enhanced solvation of excipients and reduces degradation of excipients. Preferably the formulation comprises from 0.1 to 2 w/w % propylene glycol, preferably from 0.1 to 1 w/w %, more preferably from 0.2 to 0.5% w/w, even more preferably from 0.25 to 0.4% w/w, still even more preferably about 0.34% w/w, based on the total weight of the formulation. Propylene glycol is known to be safe for human consumption, however it has been reported that prolonged exposure to high levels of inhaled propylene glycol can result in decreases in white blood cell count. Thus, in an embodiment of the present invention, the one or more pressurized containers comprise a deliverable daily dose of propylene glycol of less than 1000 mg, preferably less than 500 mg, more preferably less than 200 mg, and more preferably less than 150 mg.

Preferably the monohydric alcohol is ethanol. Ethanol has a particularly low viscosity in comparison to a glycol or glycol ether, and is therefore particularly effective at enabling the formulation to form droplets of small diameter. In addition, ethanol is cheap, relatively non-harmful and readily available. Preferably the formulation comprises from 0.5 to 1.5% w/w ethanol, preferably from 0.7 to 1.3% w/w, more preferably from 0.9 to 1% w/w, even more preferably about 0.95% w/w, based on the total weight of the formulation.

Preferably the formulation further comprises a human TAS2R bitter taste receptor agonist. The use of a human TAS2R bitter taste receptor agonist induces bronchodilation, resulting in a reduction in the levels of delivery-related coughing. Accordingly, a user is more able to tolerate the formulation since it causes very little irritation.

The human TAS2R bitter taste receptor agonist may be a naturally occurring compound or a synthetic compound. Examples of suitable naturally-occurring compounds include Absinthin, Aloin, Amarogentin, Andrographolide, Arborescin, Arglabin, Artemorin, Camphor, Cascarillin, Cnicin, Crispolide, Ethylpyrazine, Falcarindiol, Helicin, Humulone isomers, Limonin, Noscapine Papaverine, Parthenolide, Quassin, Sinigrin, and Thiamine. Examples of suitable synthetic compounds include Acesulfame K, Benzoin, Carisoprodol, Chloroquine, Cromolyn, Dapsone, Denatonium benzoate, Dimethyl thioformamide, Diphenhydramine, Divinylsulfoxide, Famotidine, Saccharin, Sodium benzoate, and Sodium cyclamate.

Preferably the human TAS2R bitter taste receptor agonist is saccharin. Saccharin is particularly effective as a human TAS2R bitter taste receptor agonist, may be readily dissolved in the formulation, is readily available and provides the formulation with a desirable taste profile.

Preferably the ratio of nicotine or a pharmaceutically acceptable derivative or salt thereof: saccharin by weight is from 12:1 to 5.5:1, preferably from 11:1 to 6:1, more preferably from 10:1 to 7:1, even more preferably from 9.5:1 to 8:1, even more preferably about 8.75:1. Lower levels of saccharin result in a formulation with an unacceptable tolerability. Higher levels of saccharin result in an acceptable tolerability but are disfavoured since they may lead to precipitates of saccharin forming in the formulation, which may cause irritation when the formulation is administered to a user or blockage when the formulation is incorporated into a simulated cigarette. Such ratios also provide the formulation with an optimised taste profile.

The propellant may be a hydrofluorocarbon, preferably a hydrofluoroalkane, even more preferably 1,1,2,2-tetrafluoroethane (HFA-134a) or 1,1,1,2,3,3-heptafluoropropane (HFC-227). Such compounds are particularly effective as propellants and have no adverse effect on the body.

The formulation may comprise at least 60% w/w propellant, preferably from 90 to 99.5% w/w, preferably from 96 to 99% w/w, more preferably from 98 to 99% w/w, based on the total weight of the formulation. The propellant is preferably liquefied.

The formulation may further comprise a flavour component. Nicotine has a bitter, long lasting taste which can often elicit a burning taste sensation. The use of a flavour component may mask this taste. Suitable flavour components include the flavour components typically added to tobacco products. Examples include carotenoid products, alkenols, aldehydes, esters and delta-lactone flavour constituents. Suitable carotenoid products include beta ionone, alpha ionone, beta-damascone, beta-damascenone, oxo-edulan I, oxo-edulan II, theaspirone, 4-oxo-beta-ionone, 3-oxo-alpha-ionone, dihydroactinodiolide, 4-oxoisophorone, safranal, beta-cyclocitral. Suitable alkenols include C₄ to C₁₀ alkenols, preferably C₅ to C₈ alkenols. Specific examples include: cis-2-Penten-1-ol, cis-2-Hexen-1-ol, trans-2-Hexen-1-ol, trans-2-Hexen-1-ol, cis-3-Hexen-1-ol, trans-3-Hexen-1-ol, trans-2-Hepten-1-ol, cis-3-Hepten-1-ol, trans-3-Hepten-1-ol, cis-4-Hepten-1-ol, trans-2-Octen-1-ol, cis-3-Octen-1-ol, cis-5-Octen-1-ol, 1-Octen-3-ol and 3-Octen-2-ol. Suitable aldehydes include benzaldehyde, glucose and cinnamaldehyde. Suitable esters include allyl hexanoate, benzyl acetate, bornyl acetate, butyl butyrate, ethyl butyrate, ethyl hexanoate, ethyl cinnamate, ethyl formate, ethyl heptanoate, ethyl isovalerate, ethyl lactate, ethyl nonanoate, ethyl valerate, geranyl acetate, geranyl butyrate, isobutyl acetate, isobutyl formate, isoamyl acetate, isopropyl acetate, linalyl acetate, linalyl butyrate, linalyl formate, methyl acetate, methyl anthranilate, methyl benzoate, methyl benzyl acetate, methyl butyrate, methyl cinnamate, methyl pentanoate, methyl phenyl acetate, methyl salicylate (oil of wintergreen), nonyl caprylate, octyl acetate, octyl butyrate, amyl acetate (pentyl acetate), pentyl hexanoate, pentyl pentanoate, propyl ethanoate, propyl isobutyrate, terpenyl butyrate, ethyl formate, ethyl acetate, ethyl propionate, ethyl butyrate, ethyl valerate, ethyl hexanoate, ethyl heptanoate, ethyl octanoate, ethyl nonanoate, ethyl decanoate, ethyl dodecanoate, ethyl myristate, ethyl palmitate. Suitable delta-lactone flavour constituents include delta-Hexalactone, delta-Octalactone, delta-Nonalactone, delta-Decalactone, delta-Undecalactone, delta-Dodecalactone, Massoia lactone, Jasmine lactone and 6-Pentyl-alpha-pyrone. Flavour components may serve to mask the taste of nicotine, which is unpleasant.

The flavour component is preferably menthol and/or vanillin. The presence of menthol, together with the saccharin, reduces the irritation experienced by a user. Preferably the formulation comprises up to 0.1% w/w menthol, preferably from 0.01% w/w to 0.08% w/w, more preferably from 0.02% w/w to 0.06% w/w, even more preferably from 0.03% w/w to 0.05% w/w, still even more preferably about 0.04% w/w, based on the total weight of the formulation.

The formulation may comprise from 0.001% w/w to 0.045% w/w nicotine or a pharmaceutically acceptable derivative or salt thereof, preferably from 0.01% w/w to 0.045% w/w, more preferably from 0.015% w/w to 0.04% w/w, even more preferably from 0.02% w/w to 0.035% w/w, still even more preferably from 0.025% w/w to 0.03% w/w, most preferably about 0.028% w/w, based on the total weight of the formulation. Such a formulation provides similar effects to a “low strength” nicotine cigarette.

The formulation may comprise from 0.04% w/w to 0.07% w/w nicotine or a pharmaceutically acceptable derivative or salt thereof, preferably from 0.045% w/w to 0.065% w/w, more preferably from 0.05% w/w to 0.06% w/w, even more preferably from 0.054% w/w to 0.058% w/w, still even more preferably about 0.056% w/w, based on the total weight of the formulation. Such a formulation provides similar effects to a “medium strength” nicotine cigarette.

The formulation may comprise from 0.065% w/w to 0.1% w/w nicotine or a pharmaceutically acceptable derivative or salt thereof, preferably from 0.07% w/w to 0.095% w/w, more preferably from 0.075% w/w to 0.09% w/w, even more preferably from 0.08% w/w to 0.088% w/w, still even more preferably about 0.084% w/w, based on the total weight of the formulation. Such a formulation provides similar effects to a “high strength” nicotine cigarette.

A particularly preferred formulation comprises, based on the total weight of the formulation:

from 0.03 to 0.05% w/w menthol, preferably about 0.04% w/w,
from 0.25 to 0.4% w/w propylene glycol, preferably about 0.34% w/w,
from 0.9 to 1% w/w ethanol, preferably about 0.95% w/w,
saccharin, and
either:
(i) from 0.025% w/w to 0.03% w/w nicotine or a pharmaceutically acceptable derivative or salt thereof, preferably about 0.028% w/w, or
(ii) from 0.054% w/w to 0.058% w/w nicotine or a pharmaceutically acceptable derivative or salt thereof, preferably about 0.056% w/w, or
(iii) from 0.08% w/w to 0.088% w/w nicotine or a pharmaceutically acceptable derivative or salt thereof, preferably about 0.084% w/w,
   the balance being HFA-134a, wherein the ratio of nicotine to saccharin by weight is from 9.5:1 to 8:1, preferably about 8.75:1. Such a formulation exhibits a particularly desirable combination of the above-described advantages.

Preferably the total solvent content, i.e. the total content of monohydric alcohol and glycol and/or glycol ether, is less than 35% w/w, preferably less than 6% w/w, more preferably from 0.1% w/w to 2.5% w/w, based on the weight volume of the formulation. Reducing the total solvent content of the formulation reduces its viscosity, meaning it is more able to form more favourable droplet sizes.

Preferably the formulation comprises less than 0.01% w/w nicotinic acid, more preferably less than 0.005% w/w, even more preferably less than 0.001% w/w nicotinic acid, based on the total weight of the formulation. Most preferably, the formulation comprises substantially no nicotinic acid. The presence of nicotinic acid may result in the formation of precipitates in the formulation.

The formulations of the first aspect may “consist of” the components recited above. The formulations of the first aspect may “consist of” the components recited above together with any unavoidable impurities. The formulation may “consist essentially of” the components recited above along with any component that has no material effect on the function of the formulation.

In a further aspect, the present invention provides a pressurised container containing the formulation for use in the methods herein described.

The pressurised container of the further aspect of the present invention may be used to release a gaseous flow of the nicotine formulation to a user. For example, the pressurised container may be provided with means for delivering the contents of the container to the lungs of a user.

The pressurised container of the present invention may be used to release the formulation to a user without the need for a separate source of energy. For example, the formulation may be released without requiring the heating of substrates, combustion of material or a battery powered electric current. As discussed above, this can result in a reduction in the levels of harmful by-products delivered to a user.

The pressurised container of the present invention may take the form of a pressurised canister, for example, a pressurised aluminium canister. The canister may be fully recyclable and/or reusable. The canister may be refilled as required by a vending machine or a larger container containing the desired formulation under a high pressure gradient. In one embodiment, the canister is a AW5052 aluminium canister.

The pressurised container may be capable of dispensing the formulation as a mixture of aerosolised droplets. Preferably, the mixture has a particle size distribution that is similar to tobacco smoke. The mixture may have the appearance of a vapour or smoke.

The pressurised container may be pressurised to a pressure of from 3×10⁵ Pa to 1.5×10⁷ Pa, preferably from 5×10⁵ Pa to 2×10⁶ Pa, more preferably from 5.5×10⁵ Pa to 1×10⁶ Pa, even more preferably at about 6×10⁵ Pa.

The pressurised container may be used to re-fill a simulated cigarette, in particular the simulated cigarette of the an aspect of the present invention described below.

The pressurised container contents may comprise from 16 to 18 mg nicotine, preferably about 17.18 mg nicotine; from 7 to 9 mg menthol, preferably about 8.176 mg menthol; from 1 to 3 mg saccharin, preferably about 1.963 mg saccharin; from 68 to 72 mg propylene glycol, preferably about 69.5 mg propylene glycol; from 190 to 200 mg ethanol, preferably about 194.2 mg ethanol; and from 18 to 22 g HFA-134a, preferably about 20.15 g HFA-134a. Alternatively, the pressurised container contents may comprise from 10 to 12 mg nicotine, preferably about 11.45 mg nicotine; from 7 to 9 mg menthol, preferably about 8.176 mg menthol; from 1.1 to 1.4 mg saccharin, preferably about 1.288 mg saccharin; from 68 to 72 mg propylene glycol, preferably about 69.5 mg propylene glycol; from 190 to 200 mg ethanol, preferably about 194.2 mg ethanol; from 18 to 22 g HFA-134a, preferably and about 20.16 g HFA-134a. Alternatively, the pressurised container contents may comprise from 5 to 7 mg nicotine, preferably about 5.73 mg nicotine; from 7 to 9 mg menthol, preferably about 8.176 mg menthol; from 0.5 to 0.8 mg saccharin, preferably about 0.654 mg saccharin; from 68 to 72 mg propylene glycol, preferably about 69.5 mg propylene glycol; from 190 to 200 mg ethanol, preferably about 194.2 mg ethanol; and from 18 to 22 g HFA-134a, preferably about 20.16 g HFA-134a. Alternatively, the pressurised container contents may comprise about from 7 to 9 mg menthol, preferably 8.176 mg menthol; from 0.1 to 0.3 mg saccharin, preferably about 0.204 mg saccharin; from 68 to 72 mg propylene glycol, preferably about 69.5 mg propylene glycol; from 190 to 200 mg ethanol, preferably about 194.2 mg ethanol; and from 18 to 22 g HFA-134a, preferably about 20.17 g HFA-134a.

The pressurised container may be used to re-fill a simulated cigarette. Such a “re-fill” container may comprise from 0.6 to 0.7 mg nicotine, preferably about 0.672 mg nicotine; from 0.2 to 0.4 mg menthol, preferably about 0.32 mg menthol; from 0.07 to 0.09 mg saccharin, preferably about 0.077 mg saccharin; from 2.5 to 2.9 mg propylene glycol, preferably about 2.72 mg propylene glycol; from 7 to 9 mg ethanol, preferably about 7.6 mg ethanol; and from 760 to 800 mg HFA-13a, preferably about 788.6 mg HFA-134a. Alternatively, such a re-fill may comprise from 0.4 to 0.5 mg nicotine, preferably about 0.448 mg nicotine; from 0.2 to 0.4 mg menthol, preferably about 0.32 mg menthol; from 0.04 to 0.06 mg saccharin, preferably about 0.051 mg saccharin; from 2.5 to 2.9 mg propylene glycol, preferably about 2.72 mg propylene glycol; from 7 to 9 mg ethanol, preferably about 7.6 mg ethanol; and from 760 to 800 mg HFA-134a, preferably about 788.9 mg HFA-134a. Alternatively, each refill may comprise from 0.1 to 0.3 mg nicotine, preferably about 0.224 mg nicotine, from 0.2 to 0.4 mg menthol, preferably about 0.32 mg menthol; from 0.01 to 0.03 saccharin, preferably about 0.026 mg saccharin, from 2.5 to 2.9 mg propylene glycol, preferably about 2.72 mg propylene glycol, from 7 to 9 mg ethanol, preferably about 7.6 mg ethanol, and from 760 to 800 mg HFA-134a, preferably about 789.1 mg HFA-134a. Alternatively, such a re-fill may comprise from 0.2 to 0.4 mg menthol, preferably about 0.32 mg menthol, from 0.007 mg to 0,009 mg saccharin, preferably about 0.008 mg saccharin, from 2.5 to 2.9 mg propylene glycol, preferably about 2.72 mg propylene glycol; from 7 to 9 mg ethanol, preferably about 7.6 mg ethanol; and from 760 to 800 mg HFA-134a, preferably about 789.4 mg HFA-134a.

The nicotine in the pressurised container contents described above may, of course, be substituted with a pharmaceutically acceptable derivative or salt thereof.

In a further aspect, the present invention provides a simulated cigarette device, also referred to herein as a simulated cigarette, comprising:
a housing;
a pressurised reservoir of inhalable formulation within the housing;
an outlet for the inhalable formulation from the reservoir and out of the housing, the outlet being configured to eject inhalable formulation therefrom in the form of droplets, at least some of the droplets having a diameter of 10 μm or less; and
an outlet valve for controlling the flow of inhalable formulation through the outlet, wherein the inhalable formulation is according to the first aspect.

For example, the outlet may be configured to eject inhalable formulation therefrom in the form of droplets, at least 1% vol of the droplets having a diameter of 10 μm or less.

Preferably the majority of the droplets (such as, for example, at least 50% vol) have a diameter of 10 μm or less, more preferably substantially all of the droplets (such as, for example, at least 90% vol) have a diameter of 10 μm or less. Preferably at least some of the droplets (such as, for example, at least 1% vol) have a diameter of 5 μm or less, preferably the majority of the droplets (such as, for example, at least 50% vol) have a diameter of 5 μm or less, more preferably substantially all of the droplets (such as, for example, at least 90% vol) have a diameter of 5 μm or less

Preferably the outlet valve is a breath-activated valve.

Preferably the simulated cigarette further comprises a capillary plug extending from the vicinity of the outlet valve into the reservoir, filling at least 50% of the volume of the reservoir and being configured to wick the inhalable formulation towards the outlet.

Preferably the simulated cigarette has a breath-activated valve and the housing has an outlet end and an opposite end and the simulated cigarette further comprises:
a formulation flow path for the flow of the formulation from the reservoir along the flow path and out of the outlet at the outlet end of the housing;
a flexible diaphragm within the housing defining an air flow path from an air inlet to an air outlet at the outlet end of the housing;
a valve element movable with the diaphragm and biased by a biasing force into a position in which it closes the formulation flow path;
wherein suction on the outlet end causes a flow through the air flow path providing a pressure differential over the valve element thereby lifting the valve element against the biasing force to open the formulation flow path; and
wherein the biasing force is arranged to close the formulation flow path once the suction ceases.

Preferably the simulated cigarette has a breath-activated valve and the breath-activated valve is a non-metered valve between the outlet and the reservoir, the breath-activated valve comprising a flow path extending from the reservoir to the outlet end, at least a portion of the flow path being a deformable tube, and a clamping member which pinches the deformable tube closed when no suction force is applied to the device and releases the tube to open the flow path when suction is applied at the outlet, to provide uninterrupted flow from the reservoir to the outlet. This simulated cigarette is referred to hereinafter as a “pinch valve” simulated cigarette.

Preferably the simulated cigarette further comprises a re-fill valve in communication with the reservoir via which the reservoir may be refilled. The simulated cigarette may be re-filled from a pressurized container according to the second aspect of the present invention.

Preferably the size of the reservoir, the pressure within the reservoir and the size of the outlet at its narrowest point are arranged so that, when the outlet valve is fully opened, the reservoir will discharge in less than 30 seconds.

Preferably the simulated cigarette is configured to eject droplets of formulation therefrom in which at least 97% vol of the droplets have a diameter of less than 10 μm, preferably at least 98% vol, more preferably at least 98.5% vol, even more preferably at least 99% vol. Droplets of diameter less than 10 μm are deposited in the lungs, meaning that a pharmacokinetic profile similar to that of a conventional cigarette is provided.

Preferably the simulated cigarette is configured to eject droplets of formulation therefrom having the following size profile:
Dv 90 of less than 20 μm, preferably less than 5 μm, more preferably less than 3 μm, even more preferably less than 2.9 μm, and/or
Dv 50 of less than 6 μm, preferably less than 0.8 μm, more preferably less than 0.7 μm, even more preferably less than 0.6 μm, and/or
Dv 10 of less than 2 μm, preferably less than 0.3 μm, more preferably less than 0.25 μm, even more preferably less than 0.2 μm.

Accordingly, in one embodiment, the simulated cigarette is configured to eject droplets with the following size profile: Dv 90<20 μm, Dv 50<6 μm and Dv 10<2 μm; preferably with the following size profile: Dv 90<5 μm, Dv 50<0.8 μm and Dv 10<0.3 μm; more preferably with the following size profile: Dv 90<3 μm, Dv 50<0.7 μm and Dv 10<0.25 μm; even more preferably with the following size profile: Dv 90<2.9 μm, Dv 50<0.6 μm and Dv 10<0.2 μm.

Such a size profile is similar to that of a conventional cigarette, meaning that the pharmacokinetic profile provided closely mimics that of a conventional cigarette.

The simulated cigarette may provide a user with a nicotine arterial Cₘₐₓ of up to 15 ng/ml, typically from 2 to 10 ng/ml, or even from 4 to 8 ng/ml. Cₘₐₓ values greater than about 2 ng/ml provide a user with a “head rush” as experienced when smoking a conventional cigarette.

The simulated cigarette may provide these Cₘₐₓ values with a tₘₐₓ of from 10 seconds to 20 minutes, typically from 5 minutes to 15 minutes, often about 12 minutes. Compared to simulated cigarette devices of the prior art, such tₘₐₓ values are closer to those exhibited by conventional cigarettes. Accordingly, the present invention more closely mimics the pharmacokinetic profile of a conventional cigarette, and is therefore particularly effective for use in NRT or as an alternative to recreational smoking of conventional cigarettes.

Preferably the simulated cigarette is configured to eject formulation therefrom at a rate of from 0.5 to 3 litres per minute. This rate is similar to the rate smoke is ejected from a conventional cigarette. Preferably the simulated cigarette is configured to provide an inhalation resistance of from 1 to 7 kPa, preferably about 4 kPa. This inhalation resistance is similar to that provided by a conventional cigarette. When the simulated cigarette is configured to have the above ejection rate and/or inhalation resistance, preferably the simulated cigarette is configured to deliver nicotine to a user at a rate of from 0.01 to 0.06 mg/ml. This is less than a conventional cigarette. However, since the habitual aspects of smoking have been mimicked by the above ejection rate and inhalation resistance, a user will experience the same level of satisfaction with a lower level of inhaled nicotine in comparison to conventional smoking cessation aids.

In a yet further aspect, the present invention provides a method of manufacturing the formulation of the present invention, the method comprising:
preparing a pre-mixture comprising a polyhydric alcohol and a glycol and/or glycol ether, and optionally a TAS2R taste receptor agonist and/or flavouring agent, wherein the ratio of polyhydric alcohol:glycol or glycol ether by weight is from 6:1 to 1:1;
adding nicotine or a pharmaceutically acceptable derivative or salt thereof to the pre-mixture to obtain a nicotine-containing mixture; and
adding a propellant to the nicotine-containing mixture.

If the nicotine is added before the polyhydric alcohol and glycol or glycol ether are combined, then precipitation of nicotine may occur. Likewise, if the formulation comprises other components, such as a flavouring component or a TAS2R taste receptor agonist, then these components should be fully mixed into the pre-mixture before the nicotine is added in order to avoid precipitation of nicotine. In particular, it has been found that when the formulation comprises menthol, the menthol should be fully dissolved into the pre-mixture before the nicotine is added in order to avoid precipitation of nicotine.

When the formulation is to include a TAS2R taste receptor agonist and/or a flavouring component, preferably the polyhydric alcohol and glycol or glycol ether are combined before the TAS2R taste receptor agonist and/or a flavouring component are added. This avoids precipitation of the flavouring component or TAS2R taste receptor agonist.

In an embodiment of the present invention, the simulated cigarette is configured to provide a user thereof with a nicotine venous Cₘₐₓ of up to 15 ng/ml and/or with a tₘₐₓ of from 10 seconds to 20 minutes.

The present invention is described by way of example in relation to the following non-limiting figures.

### FIGURES

FIG. 1 shows the mean plasma nicotine concentrations over time by treatment from the investigation in Part B;

FIG. 2 shows the mean plasma nicotine concentrations over time by treatment from the investigation in Part D;

FIG. 3: Mean craving VAS scores over time by treatment in Part B;

FIG. 4: Mean craving VAS scores over time by treatment in Part D;

FIG. 5 is an exploded perspective view of the simulated cigarette device;

FIG. 6 is an axial cross-section of the simulated cigarette;

FIG. 7 is a cross-section of the system device of the pressurised refill container and simulated cigarette in a open configuration;

FIG. 8 is a view similar to FIG. 7 with the drawer shown closed; and

FIG. 9 is an exploded perspective view of the system.

*Difference between the novel nicotine inhaler device 0.45 mg and Nicorette® Inhalator (10 mg) is significant (p=0.05)

†Difference between the novel nicotine inhaler device 0.67 mg and Nicorette® Inhalator (10 mg) is significant (p=0.05)

SE means standard error, VAS means visual analogue scale

### EXAMPLES

Sections of these examples are reproduced from a manuscript entitled “Evaluation of Pharmacokinetics, craving and smoking urges when using a novel nicotine inhaler device” submitted for publication in Nicotine and Tobacco Research, Manuscript ID: NTR-2013-681.R2.

The invention will now be described with reference to the following non-limiting examples.

### Method of Manufacture

The following starting materials were used to prepare the formulation used in the present examples:

Saccharin (Ph. Eur)
Propylene glycol (EP grade)

### Menthol (Ph Eur.)

Ethanol (100% BP, Ph. Eur.)
Nicotine (Ph. Eur)

### HFA-134a (CPMP 1994)

Starting materials were added to a mixing vessel in the following order: (i) 5.14 g saccharin, (ii) 227.0 g propylene glycol, (iii) 32.5 g menthol and (iv) 774.0 g ethanol. The mixture was then stirred at 600 rpm for 15 minutes until the menthol pellets had fully dissolved and a clear liquid was observed. 45.6 g of nicotine was then added to the mixture and stirring was continued at 600 rpm for a further 10 minutes. The resultant mixture was then added to a pressure vessel which had been purged with HFA 134a. The vessel was then sealed before being cooled until the internal temperature reached 8-12° C., at which point the temperature was maintained. Approximately 40 kg of HFA-134a was then released into the vessel before magnetic stirring at 210 rpm commenced. HFA continued to be released into the vessel until a total of 80 kg had been added, at which point the formulation was stirred at 210 rpm for a further 110 minutes. During the further stirring, the pressure was controlled to ensure that it did not exceed 4.5 bar and that the final pressure was between 3-4 bar. After stirring, the formulation was dispensed into canisters.

Varying the method by adding nicotine either before the saccharin had been added or before the menthol had fully dissolved resulted in precipitation of the nicotine.

### Droplet Size Profile

The following formulation was prepared:
0.04% w/w menthol,
0.006% w/w saccharin,
0.34% w/w propylene glycol,
0.95% w/w ethanol,
0.056% w/w nicotine, and
remainder HFA-134a.

The formulation was inserted into nine pinch valve simulated cigarettes. Five doses were emitted from each device and the droplet size profile of each was measured using a Malvern Spraytec device. The results are set out in Table 1 below:

TABLE 1
Droplet size profile.

| | MEAN | SD |
|---|---|---|
| Dv 10 (μm) | 0.198758 | 0.010005 |
| Dv 50 (μm) | 0.606342 | 0.094779 |
| Dv 90 (μm) | 2.806378 | 1.063722 |
| % vol < 10 μm | 99.02222 | 0.77704 |

### Impurities

The following formulation was prepared:
0.04% w/w menthol,
0.0032% w/w saccharin,
0.34% w/w propylene glycol and
0.95% w/w ethanol,
0.028% w/w nicotine,
remainder HFA-134a.

The formulation was then inserted into a pressurised container. The percentage volume of impurities with respect to nicotine concentration was assessed chromatographically at both the time of fill and after six months. The results are set out in Table 2 below:

TABLE 2
Stability data (inverted, 40° C./75% RH). N = 1, 2 and 3 refer to different pressurised
containers from the same batch of formulation.

| | | T = 6 months | | |
|---|---|---|---|---|
| Impurity | Initial | N = 1 | N = 2 | N = 3 |
| Anatabine | 0.02% | 0.1% | 0.1% | 0.1% |
| β-nicotyrine | Not detected | 0.2% | 0.2% | 0.2% |
| Cotinine | Not detected | 0.2% | 0.2% | 0.2% |
| Myosmine | 0.02% | 0.2% | 0.2% | 0.2% |
| Nicotine-n-oxide | Not detected | 0.3% | 0.3% | 0.3% |
| Nornicotine | Not detected | 0.1% | 0.1% | 0.1% |
| Anabasine | Not detected | Not detected | Not detected | Not detected |

### Clinical Investigation

### Part B:

A randomised, open-label, single-blind, three-way crossover study to determine the venous PK of orally inhaled nicotine at two dose levels: 0.45 mg and 0.67 mg. The 0.45 mg dose corresponds to one charge of the formulation prepared for the droplet size study in a simulated cigarette as described herein. The 0.67 mg dose corresponds to one charge of a higher strength formulation in a simulated cigarette according to a preferred embodiment of the invention (herein referred to as the device of the present invention), wherein the formulation prepared for the droplet size study is prepared using 0.084% w/w nicotine. The dosages of the present invention were compared with the Nicorette® Inhalator (10 mg).

### Part D:

A randomised, open-label, two-way crossover study to determine the venous PK of orally inhaled nicotine at one dose level (0.45 mg) delivered as for Part B via a nicotine inhaler system of the present invention compared with the Nicorette® Inhalator (10 mg).

### Participants

Healthy volunteers (male or female) aged 18-55 years were eligible to participate if they had smoked at least 10 manufactured cigarettes per day for the last year, and smoked their first cigarette within 1 hour of waking. All participants had an expired carbon monoxide level of at least 10 ppm at screening and were required to abstain from smoking for 12 hours prior to their scheduled dosing time.

Participants were excluded if they had a known or suspected history of hypersensitivity to nicotine or any other component of the inhaler or Nicorette® Inhalator. Participants were also excluded if they had a history of confirmed chronic and/or serious pulmonary disease, including asthma, or chronic obstructive pulmonary disease, a history of myocardial infarction or cerebrovascular accident, other clinically significant cardiac or renal conditions, or any co1morbidity that could place them at risk or interfere with the interpretation of the study data.

### Study Treatment

Participants were familiarized with the use of the inhaler device of the present invention (hereinafter “the inhaler device”) using a placebo formulation on the day prior to receiving active treatment. The placebo formulation was identical to the active formulation with the exception of nicotine. Study participants were blinded to the dose of nicotine administered in each part of the study. Participants inhaled the contents of one

charge of the device of the present invention in a similar way to a cigarette. All participants were instructed to inhale the dose at the same rate of one inhalation every 15 seconds over approximately 2 minutes (i.e. eight inhalations in total). The fine particle dose (<5 □m) has a specification of 160-305 □g for a formulation concentration of 0.056% w/w nicotine (i.e. 35 to 68% of the nicotine dose). The nicotine dose contained in 0.8 g of formulation (a single dose/refill of the device of the present invention) is estimated to be 0.45 mg and 0.67 mg for the 0.056% w/w and 0.084% w/w concentrations, respectively. The Nicorette® Inhalator (10 mg) is an orally inhaled NRT product and was selected as the comparator because it was the closest available presentation to the inhaler device. Participants took one inhalation of the Nicorette® Inhalator (10 mg) every 15 seconds, taking no longer than 20 minutes (i.e. 80 inhalations) to complete the dose, in line with the regimen described in the prescribing information (McNeil Products Limited, 2010). The available nicotine dose per cartridge of Nicorette® Inhalator (10 mg) is estimated to be 4 mg but is temperature dependent (McNeil Products Limited, 2010). The time at which participants started to take the first inhalation of the test product was recorded as the dose time (t=0).

In Part B, each participant received a single dose of nicotine on three consecutive days, at dose levels of 0.45 mg and 0.67 mg via the novel nicotine inhaler device, and a single dose via the Nicorette® Inhalator (10 mg).

In Part D, each participant received a single dose of nicotine via the novel nicotine inhaler device (0.45 mg) and a single dose of nicotine via the Nicorette® Inhalator (10 mg) on two consecutive days. For the device of the present invention, Part B tested the first refill, whereas Part D examined the fourth refill. During the early stages of the study, it was noted that the quantity of nicotine delivered via the novel inhaler device for the first dose (i.e. one complete refill) tended to be only approximately 70% that of subsequent doses. In Part D of the study, the fourth refill of the device of the present invention was investigated by filling and flushing the device three times, using a pump (Cole-Parmer), a Critical Flow Controller (Model TPK) and the Dose Uniformity Sampling Apparatus. Administration of the Nicorette® Inhalator (10 mg) was identical in both study parts.

### Study Assessments

### PK Analysis

PK assessment was the primary outcome measure for this study. For both Parts B and D, venous blood samples were collected 5 minutes (±1 minute) pre-dose and at 2, 4, 7, 10 minutes (±1 minute), 15, 20, 30, 40, 50, 60 minutes (±2 minutes) and 120, 180, 240 and 300 minutes (±5 minutes) post-dose (i.e. from the start of inhalation) for the measurement of plasma nicotine concentration by a liquid chromatography with tandem mass spectrometry method. The method was validated for linearity, precision and accuracy. Quality control samples at concentrations of 3.0, 7.5 and 37.5 ng/ml, as well as 37.5 ng/ml used as dilute quality control for samples of low volume (diluted 1 in 2), were used to determine inter- and intra-precision and inter- and intra-accuracy. The mean inter-run accuracy was within 1% and precision was within 5%. Derived PK parameters were summarized separately by device and dose level.

### Efficacy

Efficacy was evaluated by assessing the impact of the test devices on craving satiation and smoking urges, and their effect on aspects of nicotine withdrawal, using:

1. a visual analogue scale (VAS) to assess the level of craving of the subject based on their response to the question, ‘How strong is your craving for cigarettes?’ on a scale of 0 (no craving) to 10 (strong craving). This assessment was made pre1dose and at 4, 20, 40, 60, 120, 180, 240 and 300 minutes from the start of dosing. In Part D, additional assessments were made at 2 and 10 minutes from the start of dosing.

2. the Questionnaire of Smoking Urges (QSU1 Brief) (Cox, Tiffany, & Christen, 2001) to assess the level of craving and smoking urges based on the responses of participants to 10 statements on a scale of 1 (strongly disagree) to 7 (strongly agree). These assessments were made pre1dose and at 20, 40, 60, 120, 180, 240 and 300 minutes from the start of dosing. Component scores were determined for the ‘desire’ and ‘anticipation’ subscales. Results of the ‘anticipation’ component score of the QSU-Brief were used as a measure of nicotine craving.

### Safety

Safety and tolerability of the device of the present invention and Nicorette® Inhalator were compared. Local tolerability was an assessment of the contact area of the device of the present invention or Nicorette® Inhalator with the participant's lips. Participants were asked how the device felt in their mouth or lips, which were also assessed visually. Tolerability was an assessment of symptoms resulting from the oral inhalation from the inhalers. Any symptoms that were reported as worse than prior to dosing were recorded as adverse events (AEs). AEs were ascertained by neutral questioning and their incidence and nature were recorded and also rated by the investigator as ‘not related’, ‘possibly related’, ‘probably related’ and ‘definitely related’ to the test product. Physical examination and monitoring of vital signs (blood pressure, heart rate, respiration rate and temperature) were also conducted.

For both Parts B and D, assessments of local tolerability and vital signs were performed at 20 minutes pre-dose. Post-dose assessments measured after the start of dosing comprised: local tolerability at 4 and 20 minutes; physical examination at 300 minutes; and vital signs and AEs, SAEs and concomitant medicine at 4, 20, 40, 60, 120, 180, 240 and 300 minutes. All participants were contacted by telephone (9±2 days) following the study end and any AEs, SAEs and concomitant medicines were recorded. Treatment1 emergent AEs (TEAEs) were evaluated from the start of dosing of the test product until the safety telephone follow-up call.

### Statistical Analyses

Participants were included in the PK population if they received all of the planned doses of nicotine. Participants were included in the safety intent-to-treat population if they received one dose of nicotine. Statistical analyses were conducted using SAS® Version 9.2.

### PK Analysis

Plasma concentrations of nicotine were measured over time and derived PK parameters were summarized by device and dose level separately for both Parts B and D of the study. The PK parameters determined were the mean maximum plasma nicotine concentration (Cₘₐₓ), the mean time to maximum plasma nicotine concentration (Tₘₐₓ), and the mean area under the plasma concentration-time curve (AUC), from time zero to the end of the sample collection period (AUCₐₗₗ) and from time zero to the time of the last quantifiable concentration (AUCₗₐₛₜ), following administration of nicotine using either device. Comparative analysis between both dose levels of the novel nicotine inhaler device and the Nicorette® Inhalator (10 mg) was performed using an analysis of variance (ANOVA) with logarithmic transformation of Cmax and AUC values. Differences of p=0.05 were considered significant. PK analysis was conducted using Phoenix™ WinNonlin® Version 6.2.

### Efficacy

Craving measures and the QSU-Brief total and component scores were summarized over time by device and dose level in both study Parts B and D. A paired Student's t-test was used to analyze the difference in mean craving VAS and QSU-Brief scores by device and timepoint. Craving was also assessed from the area under the VAS score-time curve, where a lower craving AUC equates to a better craving reduction. Comparative analysis between both dose levels of the novel nicotine inhaler device and the Nicorette® Inhalator (10 mg) of efficacy was performed using an ANOVA with logarithmic transformation of AUC.

The number of participants in Parts B and D was sufficient to demonstrate equivalence at a similarity margin of 20% with 80% power using the two 1-sided 5% tests approach, assuming an underlying intra-subject coefficient of variation of 15%.

The quantity of nicotine inhaled from the device of the present invention by each participant was calculated from the weight of formulation emitted by the device and the target nicotine concentration of the formulation.

### Results

### Study Population

A total of 24 participants were randomised for Part B and a further 24 for Part D of the study. Mean ages of participants were 28.6 years (Part B) and 29.7 years (Part D), and 58% (Part B) and 54% (Part D) were male.

### Part B

All 24 participants in Part B received a single dose from the Nicorette® Inhalator (10 mg), but only 23 received a single dose of each of two dose levels of the novel nicotine inhaler device as one participant withdrew because of study restrictions. The mean (standard deviation) weights of formulation inhaled from the device of the present invention were 0.9472 g (0.2051 g) and 0.8610 g (0.3005 g), corresponding to 0.5304 mg and 0.7232 mg nicotine from the device of the present invention 0.45 mg and 0.67 mg, respectively. There were no withdrawals resulting from AEs.

### Part D

All 24 participants in Part D received single doses of the device of the present invention 0.45 mg and the

Nicorette® Inhalator (10 mg). The mean (standard deviation) weight of formulation inhaled from the device of the present invention in Part D was 0.7074 g (0.3028 g), corresponding to 0.3961 mg nicotine.

### PK Analysis

### Maximum Plasma Concentration

In Part B, the mean venous plasma nicotine concentration increased following administration of all three treatments. The mean venous plasma Cₘₐₓ following administration of the inhalation device 0.45 mg and 0.67 mg was 3.28 ng/ml and 3.92 ng/ml, respectively (see Table 3). The mean Cₘₐₓ following administration of the Nicorette® Inhalator (10 mg) was 6.57 ng/ml (Table 3).

Results for Part D were similar to those for Part B, with the Nicorette® Inhalator (10 mg) producing a higher, but later, peak than the device of the present invention. The mean Cₘₐₓ following administration of the device of the present invention 0.45 mg and Nicorette® Inhalator (10 mg) was 3.52 ng/ml and 7.63 ng/ml, respectively (Table 3).

### Time to Cₘₐₓ

The mean nicotine Cₘₐₓ calculated in Part B was higher following administration of the Nicorette® Inhalator (10 mg) than either dose from the device of the present invention (Table 3). However, the Tₘₐₓ for the Nicorette® Inhalator (10 mg) was longer than for the device of the present invention of either strength (38.0 minutes vs 18.7 and 19.2 minutes [device of the present invention 0.45 mg and 0.67 mg, respectively] in Part B, and 36.3 minutes vs 21.0 minutes [device of the present invention 0.45 mg] in Part D) (Table 3).

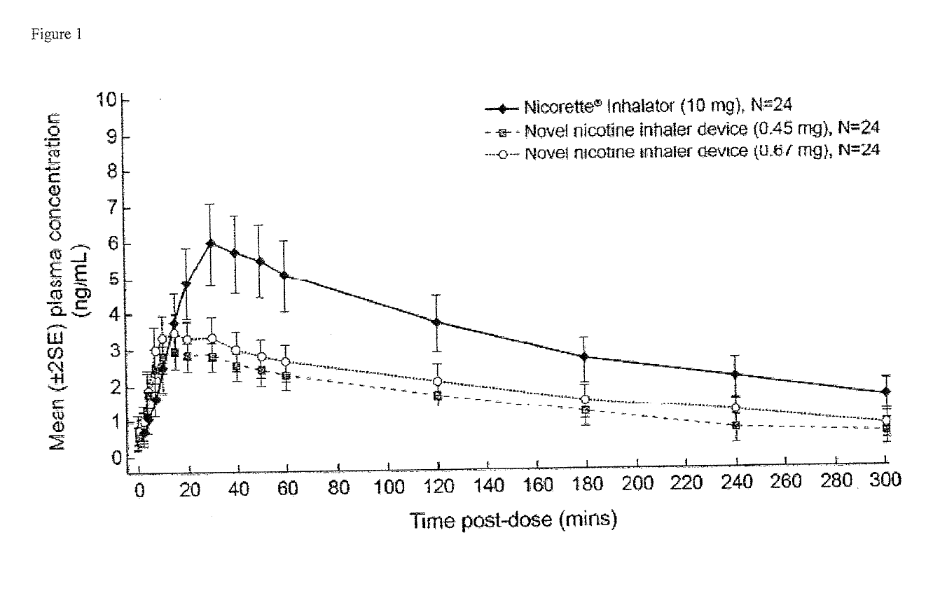

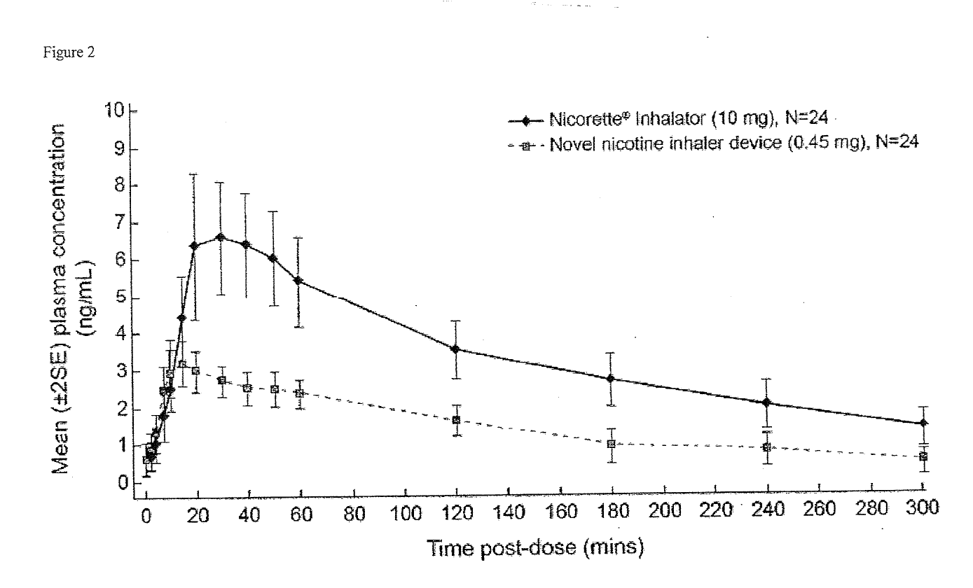

Mean plasma nicotine concentrations over time by treatment for Parts B and D are shown in FIGS. 1 and 2.

### Area Under the Concentration-Time Curve

In Part B, the mean AUC was higher for the Nicorette® Inhalator (10 mg) than for the inhalation device of either strength. Similar results were seen in Part D (Table 1).

Comparison of the relative bioavailability of nicotine between treatments in both Parts B and D indicated that the device of the present invention produced significantly lower Cₘₐₓ, AUCₗₐₛₜ and AUCₐₗₗ, and a significantly shorter Tₘₐₓ than the Nicorette® Inhalator (10 mg) (Table 1). Analysis of the AUC₀₋₁₀ from both Parts B and D confirmed the early delivery of higher amounts of nicotine from the device of the present invention compared with the Nicorette® Inhalator (10 mg).

### Efficacy

### Craving VAS Scores

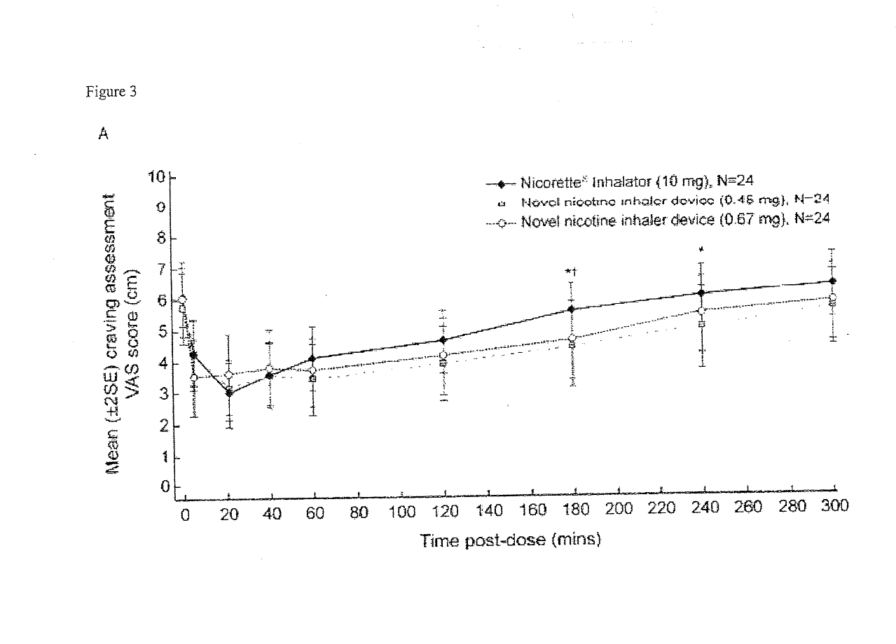

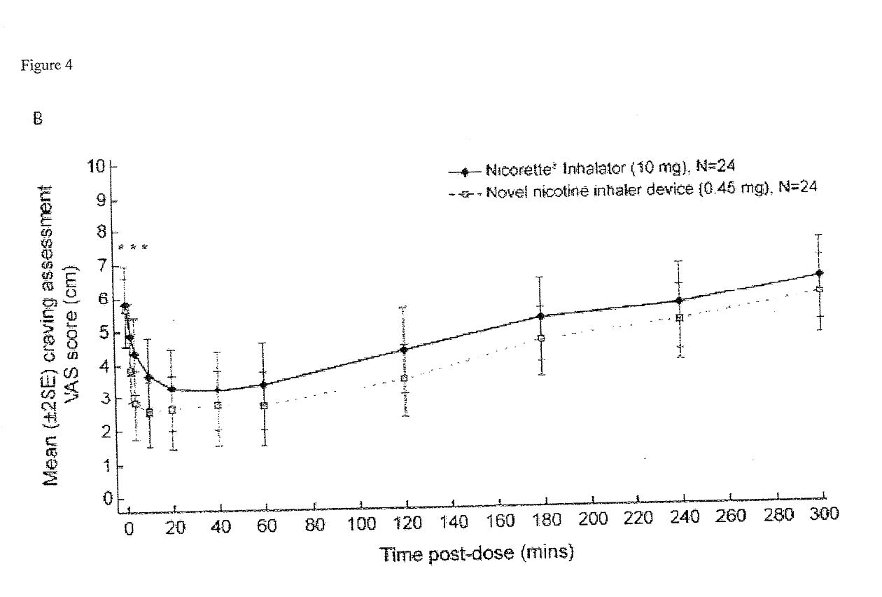

Mean craving scores assessed by the VAS were lower (higher craving relief) for the device of the present invention than the Nicorette® Inhalator (10 mg) at the majority of timepoints in both Parts B (FIG. 3) and D (FIG. 4). In Part B, the lowest score was at 20 minutes for both the device of the present invention 0.45 mg and the Nicorette® Inhalator (10 mg), and at 4 minutes for the device of the present invention 0.67 mg (FIG. 3). In Part D, the lowest mean craving VAS score was at 20 minutes for the device of the present invention 0.45 mg and at 40 minutes for the Nicorette® Inhalator (10 mg) (FIG. 4).

A comparison of the mean craving VAS scores showed that, in Part B, mean scores were lower (higher craving relief) for the device of the present invention 0.45 mg than for the Nicorette® Inhalator (10 mg) at 7 of the 8 post-dose timepoints and that this difference reached statistical significance at the 180- and 240-minute timepoints (FIG. 3). Mean scores were lower for the device of the present invention 0.67 mg than for the Nicorette® Inhalator (10 mg) at 6 of 8 post1dose timepoints, reaching statistical significance at the 180-minute timepoint (FIG. 3). In Part D, mean scores were lower for the device of the present invention 0.45 mg than for the Nicorette® Inhalator (10 mg) at all 10 post-dose timepoints, differences reaching significance at the 2-, 4-, and 10-minute timepoints (FIG. 4).

### AUC for Craving VAS Scores

In both Parts B and D, the mean AUC was lower (indicating greater craving relief) for the device of the present invention than the Nicorette® Inhalator (10 mg). In Part B, the mean (standard deviation) AUC for craving VAS score was lowest for the device of the present invention 0.45 mg (1356.3 [789.4] cm*min), followed by the device of the present invention 0.67 mg (1431.6 [769.0] cm*min). The greatest AUC was calculated for the Nicorette® Inhalator (10 mg) (1566.3 [620.4] cm*min). In Part D, the mean AUC for craving VAS score was lower for the device of the present invention 0.45 mg (1208.5 [724.4] cm*min), than for the Nicorette® Inhalator (10 mg) (1402.3 [815.2] cm*min).

A statistical comparison of craving AUC between treatments showed that in Part B, the AUC for the device of the present invention 0.45 mg was significantly lower than for the Nicorette® Inhalator (10 mg) (p=0.029); in Part D, the AUC for the device of the present invention 0.45 mg was lower than that for the Nicorette® Inhalator (10 mg) and approached statistical significance (p=0.059). These results suggest that greater relief of craving is achieved with both the medium and high doses of the device of the present invention than with the Nicorette® Inhalator (10 mg).

### QSU-Brief Score

For both parts, the mean QSU-Brief total scores were reduced (indicating craving relief) at the time of first measurement (20 minutes) for all treatment groups; similar patterns were seen for each of the QSU-Brief component scores for ‘anticipation’ and ‘desire’.

A treatment comparison of mean QSU-Brief scores showed that in Part B, total scores were statistically significantly lower for the device of the present invention 0.45 mg, than for the Nicorette® Inhalator (10 mg) at 120, 180, and 240 minutes post1dose (all p=0.05). In Part D, total scores were lower for the device of the present invention, although none of the differences were statistically significant (p>0.05).

### Safety

In Part B, a total of 87 TEAEs were reported by 23/24 (96%) participants. Of these, 79 were considered related to the study medication. Most TEAEs were mild in nature. Two TEAEs were moderate, one of which was related to study medication (local numbness, which was reported 4 minutes after administration of the inhalator and resolved within 15 minutes). There was one report of mild numbness 20 minutes post-dose with the device of the present invention 0.67 mg. For both the device of the present invention and Nicorette® Inhalator, mild tingling was the most commonly reported local tolerability symptom.

In Part D, a total of 61 TEAEs were reported by 22/24 (92%) participants. Of these, 19 reported a total of 50 TEAEs that were considered related to the study medication and all were mild. For both the device of the present invention and inhalator, mild tingling was the most commonly reported local tolerability symptom.

All AEs seen in Parts B and D were mild or moderate in nature and none were reported as severe. There were no serious AEs or deaths throughout the study, and no participants discontinued treatment because of an AE. Overall, the most common AEs were oral paresthesia, throat irritation, headache and oral hypoesthesia. In terms of local tolerability, the most common local AE reported by participants in both parts of the study was tingling of the mouth/lips.

There were no clinically significant changes in mean vital signs over time for the duration of the study.

TABLE 3
Summary of pharmacokinetic parameters by treatment

| | Part B (N = 24) | | | Part D (N = 24) | |
|---|---|---|---|---|---|
| | | Inhalation | Inhalation | | Inhalation |
| | Nicorette ® | Device-0.45 | Device-0.67 | Nicorette ® | Device-0.45 |
| | Inhaler | mg | mg* | Inhaler | mg |
| Cₘₐₓ ng/ml | 6.566 | 3.284 | 3.915 | 7.628 | 3.519 |
| | (2.965) | (1.238) | (1.640) | (4.718) | (1.378) |
| Tₘₐₓ, minutes | 38.0 | 18.7 | 19.2 | 36.3 | 21.0 |
| | (11.8) | (8.6) | (11.8) | (12.4) | (13.5) |
| AUCₗₐₛₜ, | 977.7 | 430.8 | 545.3 | 991.5 | 406.1 |
| min*ng/ml | (498.7) | (273.8) | (334.4) | (595.4) | (298.9) |
| AUCₐₗₗ, | 987.7 | 453.3 | 563.0 | 1002.6 | 433.2 |
| min*ng/ml | (487.7) | (259.0) | (322.9) | (584.5) | (284.6) |
| AUC₀₋₁₀, | 13.5 | 18.4 | 22.5 | 14.2 | 17.3 |
| min*ng/ml | (9.9) | (11.3) | (13.2) | (13.8) | (13.0) |

Data are mean (standard deviation)

All novel inhaler pharmacokinetic parameters except AUC were significantly different from the reference product (Nicorette® Inhalator) (p<0.05).

*N=23 for Part B

AUCₐₗₗ, area under the plasma concentration versus time curve from time 0 to the end of the sample collection period; AUCₗₐₛₜ area under the plasma concentration time curve from time 0 to the time of the last quantifiable concentration; AUC₀₋₁₀ area under the plasma concentration time curve from time 0 to 10 minutes; Cₘₐₓ, maximum plasma concentration; Tₘₐₓ, time to maximum concentration.

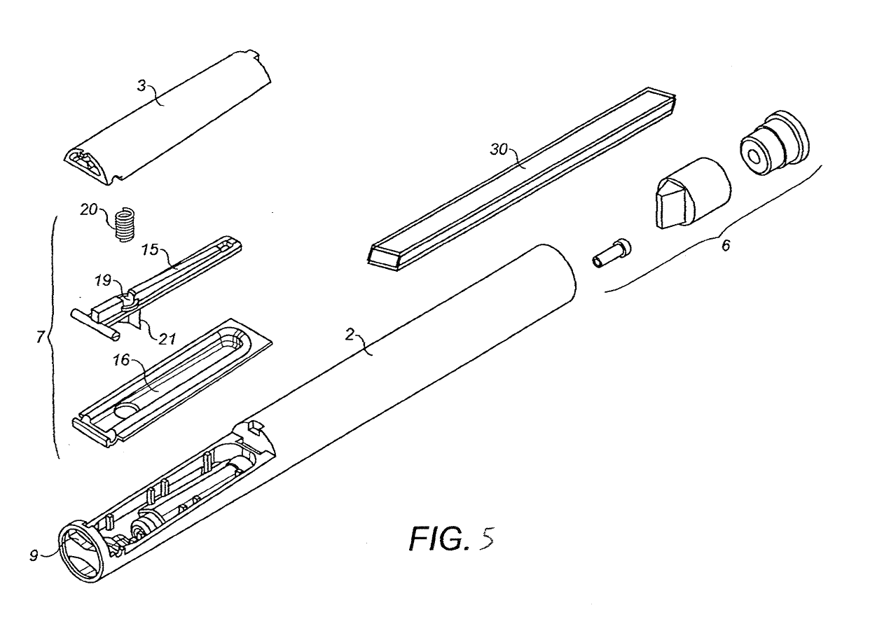

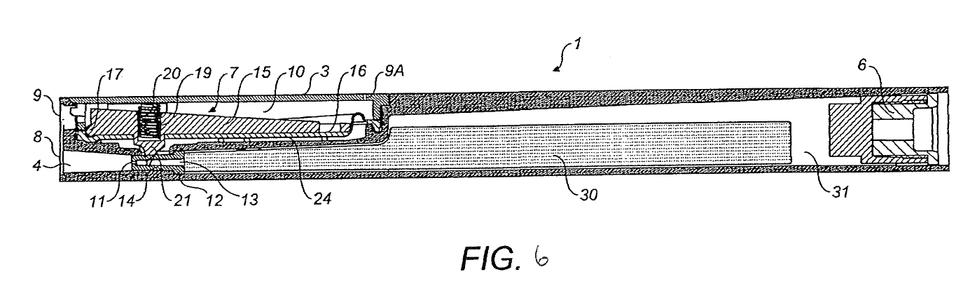

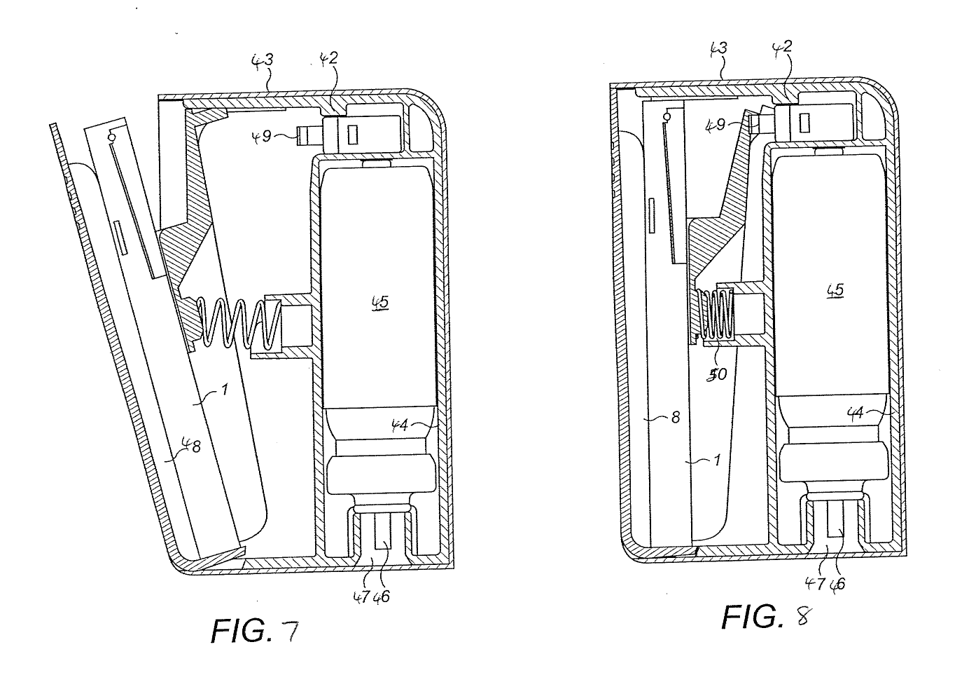

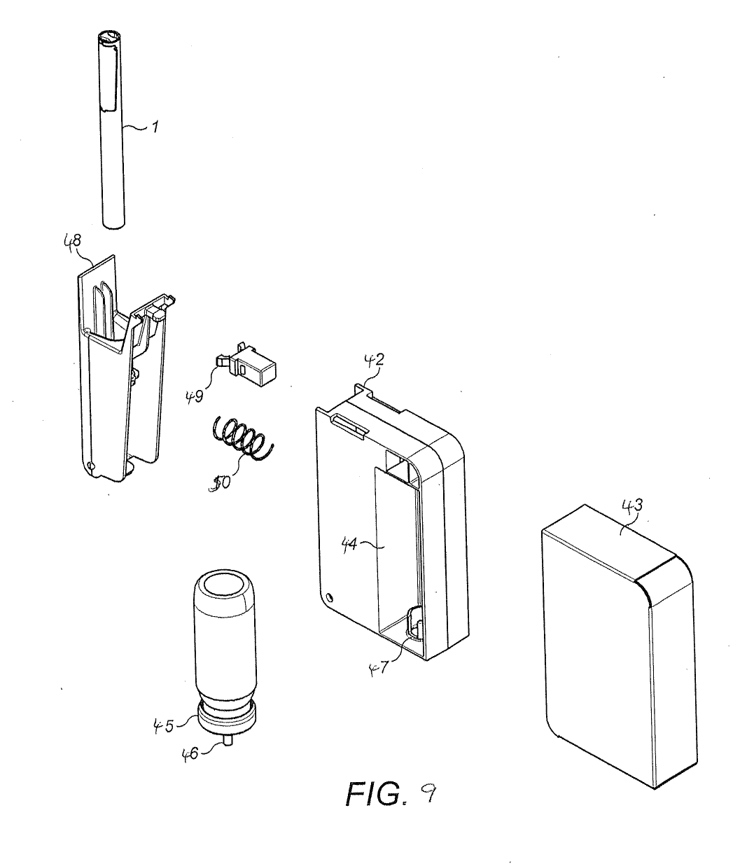

An example of the simulated cigarette device and the refill forming the simulated cigarette system will now be described with reference to FIGS. 5 to 9 with FIGS. 5 and 6 showing the simulated cigarette device and FIGS. 7 to 9 showing how this is incorporated into the refill device.

The device has a housing 1 made up of a main chassis 2 and a closure element 3 as shown in FIG. 1. This is held in place by label (not shown). Within the housing, there is a pressurised reservoir 5 containing the inhalable composition. It may be refillable as described in WO 2009/001082 through the filling valve 6, or the device may be a single use device, or may be arranged so that the reservoir 5 is a replaceable component.

The breath-activated valve 7 is positioned between an outlet end 8 and the reservoir 5. The breath-activated valve is arranged so that, when a user sucks on the outlet end 8, the breath-activated valve 7 opens to allow the inhalable composition from the reservoir 5 to be inhaled.

The housing at the outlet end has two orifices. The first of these is the suction orifice 9 which communicates with a chamber 10 as will be described in greater detail below and the second is an outlet orifice 11 from which the inhalable composition dispensed is also described in more detail below. As is apparent from FIG. 2, the outlet orifice 11 is provided on a separate component 12.

An outlet path 13 is defined between the reservoir 5 and outlet orifice 11 provided by deformable tubular element 14. This tubular element is moved between closed and open positions shown by a mechanism which will now be described.

This mechanism comprises a pivotally mounted vane 15 and a membrane 16. The pivotally mounted vane has a pivot 17 at the end closest to the outlet end 8 and a central reinforcing rib 18 running along its length and tapering away from the outlet end. At around the midpoint, the vane 15 is provided with a recess 19 for receiving a spring 20 which biases it into the closed position shown in FIG. 1 (although the deformable tubular element is shown in its undeformed state). Below the recess 19 is a jaw 21 having a triangular cross-section which is configured to apply the force provided from the vane 15 to the deformable tube 14 over a narrow area. The vane 15 is supported by the diaphragm 16 which is sealed to the housing at its ends 22, 23.

This seals off the chamber 10 other than to the suction orifice 9 and an air vent that is located in the closure element 3.

The underside 24 of the membrane 16 is open to atmospheric pressure as a leakage path exists through the housing 1 which is not shown in the drawings as it extends around the outlet path 1 and is therefore not shown in the plane of FIG. 1.

When a user sucks on the outlet end 8, the suction is communicated by the suction orifice 9 to the chamber 10 thereby lowering the pressure in this chamber. This causes the vane 15 to be lifted against the action of the spring 20 deforming the diaphragm and lifting the jaw 21 to allow the deformable tube to open, thereby allowing the inhalable composition from the reservoir 5 along outlet path 13 through the deformable tube 14 and out through the outlet orifice 11. The degree of suction applied by the user will determine the extent to which the vane 15 moves and therefore the amount of composition that the user receives. As soon as a user stops sucking, atmospheric pressure will return to the chamber 10 via the suction orifice 9 and the spring 20 will return the vane thereby pinching the tube 14 closed.

Within the reservoir is a capillary plug 30. As is apparent from FIG. 2, this extends for substantially the entire length of the reservoir, although there is a gap 51 between the end of the rod and the refill valve 6. As can also be seen in FIG. 2, the plug 30 does not fill the entire cross-section of the reservoir, at least in the region away from the valve 7. Instead, a gap 32 exists along the top surface of the rod. This allows refill material to pass along this gap and be absorbed along the length of the rod, rather than all of the refill liquid having to enter through the end of the rod.

The remainder of the simulated smoking system is the refill device. This comprises a main housing portion 42 which is a plastics moulding. This is surrounded by a thin card sleeve 43 on which is printed various information such as promotional information. The size of the housing is preferably similar to the size of a cigarette pack and may be adjusted to suit particular sizing formats, e.g., to be the size of pack of 10 or 20 cigarettes. The housing 42 has a recess 44 in which is contained refill canister 45 of pressurised refill gas. The canister 45 has an outlet stem 46 at its lowermost end. With the canister 45 in the recess 44, the nozzle 46 sits above refill outlet orifice 47. The simulated cigarette device 1 is refilled by being pressed against the refill outlet orifice 47 as described in detail below. The canister may instead simply be a stand alone cylinder which could then have a larger capacity as it is not constrained by needing to fit within a cigarette pack sized housing.

In general terms, the housing 42 is divided into two halves with one half containing the refill gas canister 45 and the other part containing a hinged drawer 48 with a release mechanism 49 biased by a spring 50. This arrangement forms the subject of WO 2011/095781.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

1. A simulated cigarette system comprising:
an orally inhalable formulation comprising nicotine or a pharmaceutically acceptable salt thereof and a propellant
a pressurized refill container containing the inhalable formulation,
a simulated cigarette for pulmonary administration of the formulation, said simulated cigarette having a housing, a reservoir configured to receive and contain a charge of inhalable formulation from the pressurized container, and an outlet valve, and
wherein said refill container contains 2 mg to 20 mg of nicotine or a pharmaceutically acceptable salt thereof.

2. A system according to claim 1, wherein the pressurized container comprises approximately 15-30 charges for the simulated cigarette.

3. A system according to claim 1 or claim 2, wherein the second and subsequent charges comprise approximately 0.1-1 mg nicotine or a pharmaceutically acceptable salt thereof.

4. A system according to claim 3, wherein the second and subsequent charges comprise approximately 0.2-0.7 mg nicotine or a pharmaceutically acceptable salt thereof.

5. A system according to claim 1, wherein the first charge comprises less than approximately 1 mg nicotine or a pharmaceutically acceptable salt thereof, preferably less than approximately 0.43 mg, more preferably approximately 0.02-0.3 mg.

6. A system according to claim 1, wherein the propellant is HFA, preferably HFA-134a.

7. A system according to claim 1 wherein the formulation further comprises:
a monohydric alcohol; and
a glycol and/or glycol ether,
characterized in that the ratio of monohydric alcohol:glycol or glycol ether by weight is from 6:1 to 1:1.

8. A system according to claim 7, wherein the monohydric alcohol is ethanol.

9. A system according to claim 7 or claim 8, wherein the glycol and/or glycol ether is propylene glycol.

10. A system according to claim 1, wherein the formulation comprises, based on the total weight of the formulation:
from 0.03-0.05% w/w menthol,
from 0.25-0.4% w/w propylene glycol,
from 0.9-1% w/w ethanol,
saccharin, and
either
(i) from 0.025% w/w to 0.03% w/w nicotine or a pharmaceutically acceptable salt thereof, or
(ii) from 0.054% w/w to 0.058% w/w nicotine or a pharmaceutically acceptable salt thereof, or
(iii) from 0.08% w/w to 0.088% w/w nicotine or a pharmaceutically acceptable salt thereof,
the balance being HFA-134a, wherein the ratio of nicotine or a pharmaceutically acceptable salt thereof to saccharin is from 9.5:1 to 8:1% w/w.

11. A system according to claim 1, wherein the outlet of the simulated cigarette is configured to eject inhalable formulation therefrom in the form of droplets, at least some of the droplets having a diameter of 10 μm or less.

12. A system according to claim 1, wherein the outlet valve is a breath-actuated valve.

13. A system according to claim 1, wherein the simulated cigarette further comprises a capillary plug extending from the vicinity of the outlet valve into the reservoir, filling at least 50% of the volume of the reservoir and being configured to wick the inhalable composition towards the outlet.

14. A method of relieving or preventing nicotine craving associated with tobacco dependence in a subject, said method comprising the step of
administering to the subject an orally inhalable formulation comprising nicotine or a pharmaceutically acceptable salt thereof and a propellant,
wherein a daily dose of inhalable formulation is provided in one or more pressurized containers,
wherein the inhalable formulation is for pulmonary administration using a simulated cigarette, said simulated cigarette having a housing, a reservoir configured to receive and contain a charge of inhalable formulation from the pressurized container, and an outlet valve,
wherein said daily dose of inhalable formulation comprises a deliverable daily dose of less than 60 mg of nicotine or a pharmaceutically acceptable salt thereof and each of the one or more pressurized containers contains from about 2 mg to about 20 mg nicotine or a pharmaceutically acceptable salt thereof.

15. A method of relieving or preventing withdrawal symptoms associated with tobacco dependence in a subject, said method comprising the step of
administering to the subject an orally inhalable formulation comprising nicotine or a pharmaceutically acceptable salt thereof and a propellant,
wherein a daily dose of inhalable formulation is provided in one or more pressurized containers,
wherein the inhalable formulation is for pulmonary administration using a simulated cigarette, said simulated cigarette having a housing, a reservoir configured to receive and contain a charge of inhalable formulation from the pressurized container, and an outlet valve,
wherein said daily dose of inhalable formulation comprises a deliverable daily dose of less than 60 mg of nicotine or a pharmaceutically acceptable salt thereof and each of the one or more pressurized containers contains from about 2 mg to about 20 mg nicotine or a pharmaceutically acceptable salt thereof.

16. A method of reducing or preventing consumption of inhaled tobacco smoke in a subject, said method comprising the step of
administering to the subject an orally inhalable formulation comprising nicotine or a pharmaceutically acceptable salt thereof and a propellant,
wherein a daily dose of inhalable formulation is provided in one or more pressurized containers,
wherein the inhalable formulation is for pulmonary administration using a simulated cigarette, said simulated cigarette having a housing, a reservoir configured to receive and contain a charge of inhalable formulation from the pressurized container, and an outlet valve,
wherein said daily dose of inhalable formulation comprises a deliverable daily dose of less than 60 mg of nicotine or a pharmaceutically acceptable salt thereof and each of the one or more pressurized containers contains from about 2 mg to about 20 mg nicotine or a pharmaceutically acceptable salt thereof.
